(19) United States
(12) Patent Application Publication
(10) Pub. No.: US 2023/0036777 A1
(43) Pub. Date: Feb. 2, 2023

(54) TINTING CONDITIONER HAVING IMPROVED DYEING PERFORMANCE

(71) Applicant: Henkel AG & Co. KGaA, Duesseldorf (DE)

(72) Inventors: Camille GROSJACQUES, Hamburg (DE); Katharina HEIDENREICH, Hamburg (DE)

(73) Assignee: Henkel AG & Co. KGaA, Duesseldorf (DE)

(21) Appl. No.: 17/785,880

(22) PCT Filed: Nov. 17, 2020
(86) PCT No.: PCT/EP2020/082331

§ 371 Date: Jun. 15, 2022

(30) Foreign Application Priority Data
Dec. 16, 2019 (DE) 10 2019 219 650.0

(51) Int. Cl. A61K 8/44; A61K 8/891; A61K 8/34; A61K 8/19; A61K 8/92; A61Q 5/10

(52) U.S. Cl. CPC A61K 8/44; A61K 8/891; A61K 8/342; A61K 8/19; A61K 8/922; A61Q 5/10; A61K 2800/432; A61K 2800/30

(57) ABSTRACT

The present disclosure is in the field of cosmetics and relates to tinting conditioners for the simultaneous semi-permanent coloring and conditioning of hair, comprising at least one direct dye and a combination of at least one fatty alcohol, at least one alkylamidoalkyl betaine and at least one water-soluble magnesium salt in an amount such that the content of Mg′ ions is from about 0.005 to about 1.000% by weight, based on the weight of the tinting conditioner. These tinting agents are suitable for the care and conditioning of hair, especially for smoothing the roughened surface of the hair and split ends, and at the same time particularly good for improving the color fastness of coloring on the hair.

### CROSS-REFERENCE TO RELATED APPLICATION

This application is a U.S. National-Stage entry under 35 U.S.C. § 371 based on International Application No. PCT/EP2020/082331, filed Nov. 17, 2020, which was published under PCT Article 21(2) and which claims priority to German Application No. 102019219650.0, filed Dec. 16, 2019, which are all hereby incorporated in their entirety by reference.

### TECHNICAL FIELD

The present disclosure is in the field of cosmetics and relates to tinting conditioners for the simultaneous semi-permanent coloring and conditioning of hair, comprising at least one direct dye and a combination of at least one fatty alcohol, at least one alkylamidoalkyl betaine and at least one water-soluble magnesium salt in an amount such that the content of Mg²⁺ ions is from about 0.005 to about 1.000% by weight, based on the weight of the tinting conditioner. These tinting agents are suitable for the care and conditioning of hair, especially for smoothing the roughened hair surface and split ends and at the same time particularly well for

Improving the color fastness of the coloring on the hair and/or

Increasing the chromaticity of the coloring on the hair and/or

Increasing the fixation of dyes on the hair and/or

Increasing the color intensity of the coloring on the hair.

### BACKGROUND

The special combination of ingredients in the fatty alcohol emulsion as cosmetic carrier as contemplated herein, in particular the combination of amphoteric surfactant and magnesium salt according to claim 1, achieved very good dyeing results in terms of color fastness as well as dyeing properties, such as chromaticity and color intensity. In the existing art, agents for semi-permanent hair coloring containing an alkylamidoalkyl betaine, in particular an alkylamidopropyl betaine, were already known. The content of water-soluble magnesium salt, such as in particular magnesium chloride or trimagnesium dicitrate, makes a surprising contribution to the improvement of the fastness properties of the coloration as well as to the hair conditioning properties.

Tinting conditioners are currently an extremely popular product group among consumers, as they satisfy the desire to refresh an already existing coloration, which is usually a permanent oxidation coloration, without damaging the hair and, on the contrary, still providing it with special care. In addition, this group of products combines two functions in one product, coloring and conditioning the hair. Hair conditioners as well as tinting agents, i.e. direct dyes, require a certain exposure time. For time-saving reasons, it is attractive to the consumer to have both functions combined in a single product.

In addition, semi-permanent dyeing with direct dyes allows hair color to be varied more frequently due to the faster washout compared to oxidative dyes.

The basic condition for solving the tasks set was to use a fatty alcohol-containing emulsion as the basis for the tinting conditioner as contemplated herein in order to take advantage of the proven hair conditioning properties of this carrier material. During product development, a balance had to be found between acceptable usage properties, in particular good washability of the product after the exposure time, good color retention on the hair fibers, long-lasting coloration, high color fastness and, in addition, good hair conditioning properties, because many consumers use such a tinting conditioner on their hair, which is already pre-damaged by oxidative hair coloring or bleaching.

Changing the shape and color of keratinous fibers, especially hair, is an important area of modern cosmetics. This allows the appearance of the hair to be adapted to current fashion trends as well as to the individual wishes of the person. To change the color of the hair, the specialist knows various coloring systems, depending on the requirements of coloring. For permanent, intensive dyeings with good fastness properties and good gray coverage, oxidation dyes are usually used. Such colorants usually contain oxidation dye precursors, so-called developer components and coupler components, which form the actual dyes under the influence of oxidizing agents such as hydrogen peroxide. Oxidation dyes are exemplified by excellent, long-lasting coloring results, but are also associated with a certain degree of hair damage.

If the user wants to reduce hair damage or change the hair color only temporarily, she/he can resort to dyes based on direct dyes. In this method, already fully formed dyes diffuse from the colorant into the hair fiber. Compared to oxidative hair dyeing, the dyeings obtained with direct dyes have lower durability and faster washout. Gray coverage that can be obtained with direct dyes also usually needs improvement. However, the advantage is the less damage to the hair of coloring with direct dyes.

Depending on the desired color result, the specialist uses direct dyes of different dye classes. The existing direct dyes belong, for example, to the class of nitro dyes, anthraquinone dyes, azo dyes, triarylmethane dyes or methine dyes. All these dye classes should meet a certain requirement profile for use in the cosmetics sector. Thus, direct dyes should provide an intensive dyeing result and have as good fastness properties as possible. The color result obtained with direct dyes should be influenced as little as possible by environmental influences, i.e. the dyes should, for example, have good color fastness, lightfastness and rub fastness. Chemical influences to which the keratin fibers may be exposed after the dyeing process (such as perms) should also change the color result as little as possible.

Direct dyes can be divided into anionic, cationic and non-ionic direct dyes. In cosmetic compositions, these interact in different ways with the other ingredients of the compositions. Surface-active agents such as surfactants and emulsifiers have a major influence here. In cosmetic compositions, these are intended to remove impurities from the keratinous fibers and/or emulsify components of the composition itself. However, depending on the selection of the surface-active substance, interactions between these and the direct dyes can also occur, which sometimes significantly worsens the dyeing result compared to agents that do not contain surface-active substances.

Surfactants or emulsifiers are amphiphilic (bifunctional) compounds comprising at least one hydrophobic and at least one hydrophilic moiety. The hydrophobic moiety is usually a hydrocarbon chain. The hydrophilic moiety may have a negative charge, a positive charge, a negative and a positive charge, or no charge, depending on the type of surfactant.

In anionic surfactants, the hydrophilic moiety includes at least one negatively charged hydrophilic head group. Anionic surfactants contain only negative charges.

Cationic surfactants are surfactants, i.e. surface-active substances, each with one or more positive charges. Cationic surfactants contain only positive charges.

Zwitterionic (amphoteric) surfactants comprise at least one negatively charged and at least one positively charged group in the hydrophilic moiety. These are spatially separated from each other and are present next to each other, with the surfactant being electrically neutral overall.

In addition, there are non-ionic (non-ionogenic) surfactants, which are exemplified by the absence of electric charges in the molecules.

### BRIEF SUMMARY

Cosmetic agents for dyeing and caring for hair are provided, and methods for the same. In an exemplary embodiment, a cosmetic agent includes: (a) a direct dye; (b) an alkylamidoalkyl betaine; (c) a nonionic ethoxylate surfactant; (d) a linear l-alkanol; (e) a water-soluble magnesium salt; and (f) water. With weight percentages based on a total weight of the agent, the alkylamidoalkyl betaine is present in the agent in an amount of from about 0.05 to about 3.0% by weight. The nonionic ethoxylate surfactant is present in the agent in an amount of from about 0.2 to about 1.5% by weight, and the linear l-alkanol is present in the agent in an amount of from about 5 to about 15% by weight. The water-soluble magnesium salt is present in the agent such that the content of Mn′ ions in the agent is from about 0.005 to about 1.0% by weight. The water is present in an amount of from about 74 to about 86% by weight. The linear l-alkanol has from about 8 to about 24 carbon atoms. The agent is free of alkyl glycosides, oxidation dye precursors, and oxidation agents. A total surfactant content of the agent is from about 0.5 to about 6.0% by weight.

A method for dyeing and caring for hair is provided in another embodiment. The method includes applying a cosmetic agent to the hair, where the cosmetic agent includes: (a) a direct dye; (b) an alkylamidoalkyl betaine; (c) a nonionic ethoxylate surfactant; (d) a linear l-alkanol; (e) a water-soluble magnesium salt; and (f) water. With weight percentages based on a total weight of the agent, the alkylamidoalkyl betaine is present in the agent in an amount of from about 0.05 to about 3.0% by weight. The nonionic ethoxylate surfactant is present in the agent in an amount of from about 0.2 to about 1.5% by weight, and the linear l-alkanol is present in the agent in an amount of from about 5 to about 15% by weight. The water-soluble magnesium salt is present in the agent such that the content of Mn²⁺ ions in the agent is from about 0.005 to about 1.0% by weight. The water is present in an amount of from about 74 to about 86% by weight. The linear l-alkanol has from about 8 to about 24 carbon atoms. The agent is free of alkyl glycosides, oxidation dye precursors, and oxidation agents. A total surfactant content of the agent is from about 0.5 to about 6.0% by weight. The agent is exposed to the hair for an exposure time of from about 1 to about 30 minutes, and then rinsed out of the hair.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The present disclosure was therefore based on the task of providing a conditioning hair colorant for semi-permanent coloring with improved color fastness.

Surprisingly, it has now been found that the color fastness of semi-permanent dyeings with at least one direct-acting dye can be improved out of a fatty alcohol-in-water emulsion when at least one alkylamidoalkyl betaine is present in a total amount of about 0.05 to about 3% by weight and at least one water-soluble magnesium salt is present in such an amount that the content of Mg²⁺ ions is about 0.005 to about 1.000% by weight, each based on the weight of the tinting or coloring agent.

A first object of the present disclosure is a cosmetic composition for the simultaneous semi-permanent coloring and conditioning of hair according to claim 1, comprising
(a) at least one direct dye,
(b) at least one alkylamidoalkyl betaine in a total amount of about 0.05-3% by weight,
(c) at least one nonionic ethoxylate surfactant, in a total amount of about 0.2-1.5% by weight, preferably about 0.4-1.2 wt %, particularly preferably about 0.6-0.9% by weight,
(d) at least one linear l-alkanol having about 8-24 carbon atoms in a total amount of about 5-15% by weight, preferably about 8-13% by weight more preferably about 10-12% by weight,
(e) at least one water-soluble magnesium salt in such an amount that the content of Mg2+ ions is about 0.005 to about 1.000 wt %, preferably about 0.008 to about 0.500 wt %, more preferably about 0.010 to about 0.200 wt %, especially preferably about 0.013 to about 0.100 wt %, exceptionally preferably about 0.018 to about 0.050 wt %,
(f) about 74-86 wt % water,
   wherein no alkyl glycoside, no oxidation dye precursor and no oxidizing agent are present, the total surfactant content being about 0.5-6% by weight, preferably about 1-5% by weight, particularly preferably about 1.5-3% by weight, and
   where all quantities in % by weight refer to the weight of the cosmetic product.

Keratinous fibers, or keratin fibers, are understood to mean furs, wool, feathers and, in particular, human hair. Although the agents are primarily suitable for dyeing keratin fibers, in principle there is nothing to prevent their use in other areas as well.

The terms “agent for semi-permanent hair dyeing”, “dyeing agent” and “tinting agent” are used to refer to dyeing agents that dye hair on the basis of direct dyes without the influence of an oxidizing agent.

As a first essential ingredient, the composition as contemplated herein contains at least one direct dye (a).

The direct dye(s) (a) is/are preferably present in a total amount of from about 0.001 to about 7% by weight, more preferably from about 0.01 to about 5.5% by weight, more preferably from about 0.08 to about 3.4% by weight, more preferably from about 0.1 to about 2% by weight, more preferably from about 0.3 to about 1.5% by weight and further preferably from about 0.6 to about 1% by weight, in each case based on the weight of the colorant.

Direct dyes (a) can be divided into anionic, cationic and non-ionic direct dyes. The direct dyes are usually selected from the nitrophenylenediamines, the nitroaminophenols, the azo dyes, the anthraquinones, the triarylmethane dyes or the indophenols and their physiologically compatible salts.

In the case of dyes that carry exclusively anionic charges, experts refer to them as acid dyes. The terms anionic dye and acid dye are therefore used synonymously for the purposes of this present disclosure. Anionic dyes or acid dyes are direct dyes which have at least one carboxylic acid group (—COOH) and/or at least one sulfonic acid group (—SO₃H). Depending on the pH, the protonated forms (—COOH, —SO₃H) of the carboxylic acid or sulfonic acid groups are in equilibrium with their deprotonated forms (—COO⁻.—SO₃⁻). As pH decreases, the proportion of protonated forms increases. If direct dyes are used in the form of their salts, the carboxylic acid groups or sulfonic acid groups are present in deprotonated form and are neutralized with corresponding stoichiometric equivalents of cations (such as Na cation or K cations) to maintain electroneutrality. An anionic dye does not carry cationic charges.

Suitable acid dyes may include, for example, one or more compounds selected from the following group: Acid Yellow 1 (D&C Yellow 7, Citronin A, Ext. D&C Yellow No. 7, Japan Yellow 403, CI 10316, COLIPA no B001), Acid Yellow 3 (COLIPA no: C 54, D&C Yellow No 10, Quinoline Yellow, E104, Food Yellow 13), Acid Yellow 9 (CI 13015), Acid Yellow 17 (CI 18965), Acid Yellow 23 (COLIPA no C. 29, Covacap Jaune W 1100 (LCW), Sicovit® Tartrazine 85 E 102 (BASF®), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA no C015), Acid Orange 10 (CI 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; no sodium salt; Brown No. 201; RESORCIN BROWN; ACID ORANGE 24; Japan Brown 201; D & C Brown No. 1), Acid Red 14 (CI14720), Acid Red 18 (E124, Red 18; CI 16255), Acid Red 27 (E 123, CI 16185, C Red 46, Real Red D, FD&C Red No. 2, Food Red 9, Naphthol Red S), Acid Red 33 (Red 33, Fuchsia Red, D&C Red 33, CI 17200), Acid Red 35 (CI18065), Acid Red 51 (CI 45430, Pyrosine B, Tetraiodofluorescein, Eosin J, Iodeosin), Acid Red 52 (CI 45100, Food Red 106, Solar Rhodamine B, Acid Rhodamine B, Red no 106 Pontacyl Brilliant Pink), Acid Red 73 (CI 27290), Acid Red 87 (Eosin, CI 45380), Acid Red 92 (D&C Red; Red 104; AKA231; RED 28; SUREDYE; 11969 Red; PHLOXINE; CI 45405; CI 45410; EOSINE B); Acid Red 95 (CI 45425, Erythrosine, Simacid Erythrosine Y), Acid Red 184 (CI 15685), Acid Red 195; Pigment Red 57:1 (E180; D&CRED7; CI 15850; Rubies 4BN; CI 15850:1; PIGMENT RED 57; Litholrubine BK; LITHOLRUBINE RB; LITHOLRUBINE BCA; Lithol Rubies B); Acid Violet 43 (Jarocol Violet 43, Ext. D&C Violet no 2, CI 60730, COLIPA n° C.063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent Blue AE, Amido Blue AE, Erioglaucin A, CI 42090, CI Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (CI 42100), Acid Green 22 (CI 42170), Acid Green 25 (CI 61570, Japan Green 201, D&C Green No. 5), Acid Green 50 (Brilliant Acid Green BS, CI 44090, Acid Brilliant Green BS, E 142), Acid Black 1 (Black no 401, Naphthalene Black 10B, Amido Black 10B, CI 20470, COLIPA no B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Brown 1, Bromophenol Blue and/or Tetrabromophenol Blue and mixtures thereof.

In a further particularly preferred embodiment, a composition as contemplated herein is exemplified in that it comprises (a) at least one anionic direct dye selected from the group of Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red 18, Acid Red 27, Acid Red 33, Acid Red 35, Acid Red 51, Acid Red 52, Acid Red 73, Acid Red 87, Acid Red 92, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D&C Yellow 7, D&C Yellow 8, D&C Orange 4, D&C Green 5, D&C Orange 10, D&C Orange 11, D&C Red 21, D&C Red 27, D&C Red 33, D&C Violet 2 and D&C Brown 1.

Very preferably, an agent as contemplated herein is exemplified in that it comprises at least one anionic direct dye (a) from the group of Acid Orange 7, Acid Red 33, Acid Red 92; Acid Violet 43, Acid Blue 9, Acid Blue 62 and Acid Black 1.

The anionic direct dye(s) (a) are preferably present in a total amount of from about 0.001 to about 7% by weight, preferably from about 0.01 to about 5.5% by weight, more preferably from about 0.1 to about 3.4% by weight and particularly preferably from about 0.3 to about 2% by weight, in each case based on the weight of the colorant.

Further colorants preferred as contemplated herein are exemplified in that they contain at least one cationic direct dye as the direct dye (a). Dyes that carry only cationic charges are usually also referred to as basic dyes.

Examples of suitable basic (cationic) dyes include: Basic Blue 6 (CI-No. 51.175), Basic Blue 7 (CI-No. 42,595) Basic Blue 9 (CI-No. 52.015), Basic Blue 26 (CI-No. 44,045), Basic Blue 41 (CI-No. 11,154), Basic Blue 99 (CI-No. 56.059), HC Blue 15, HC Blue 16 (bluequat bromide), Cationic Blue 347, Basic Brown 4 (CI-No. 21.010), Basic Brown 16 (CI-No. 12,250), Basic Brown 17 (CI-No. 12,251), Natural Brown 7 (CI-No. 75,500), Basic Green 1 (CI-No. 42.040), Basic Red 2 (CI-No. 50,240), Basic Red 22 (CI-No. 11.055), Basic Red 51, Basic Red 76 (CI-No. 12,245), Basic Violet 1 (CI-No. 42,535), Basic Violet 2, Basic Violet 3 (CI-No. 42,555), Basic Violet 10 (CI-No. 45,170), Basic Violet 14 (CI-No. 42,510), Basic Yellow 57 (CI-No. 12,719), Basic Yellow 87 and Basic Orange 31, as well as combinations of the above dyes.

One or more dyes from the group HC Blue 15, HC Blue 16 (bluequat bromide), Cationic Blue 347, Basic Violet 2, Basic Red 51, Basic Red 76, Basic Yellow 57, Basic Yellow 87, Basic Orange 31, and Basic Brown 17 have proved particularly suitable.

Very preferably, an agent as contemplated herein is exemplified in that it contains at least one cationic direct dye (a) from the group of HC Blue 15, HC Blue 16, Basic Red 76, Basic Yellow 57, Basic Orange 31, Basic Red 51 and Basic Brown 17.

The cationic direct dye(s) (a) are preferably present in a total amount of from about 0.001 to about 7% by weight, preferably from about 0.01 to about 5.5% by weight, more preferably from about 0.1 to about 3.4% by weight and particularly preferably from about 0.3 to about 2% by weight, in each case based on the weight of the colorant.

Further colorants preferred as contemplated herein are exemplified in that they contain at least one non-ionic direct dye as the direct dye (a). This may be selected, for example, from the group HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, HC Blue 15, Disperse Blue 3, HC Violet 1, Disperse Violet 1, Disperse Violet 4, Disperse Black 9, 1,4-diamino-2-nitrobenzene, 2-amino-4-nitrophenol, 1,4-bis(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3-nitrophenol, 4-nitro-ophenylenediamine, 6-nitro-1,2,3,4-tetrahydroquinoxaline, 2-hydroxy-1,4-naphthoquinone, picramic acid and its salts, N,N′-bis-(2-hydroxyethyl)-2-nitro-p-phenylenediamine, 2-amino-6-chloro-4-nitrophenol, 4-ethylamino-3-nitrobenzoic acid and 2-chloro-6-ethylamino-4-nitrophenol.

Very preferably, an agent as contemplated herein is exemplified in that it comprises at least one nonionic direct dye (a) from the group of HC Red 1, HC Red 3, HC Red 7, HC Red 13, HC Orange 1, HC Yellow 2, HC Yellow 13, HC Blue 2, HC Blue 11, HC Blue 12, HC Blue 14, HC Violet 2, Disperse Violet 1, 4-[(3-hydroxypropyl)amino]-3-nitrophenol and 4-amino-3-nitrophenol.

The nonionic direct dye(s) (a) are preferably present in a total amount of from about 0.001 to about 7% by weight, preferably from about 0.01 to about 5.5% by weight, more preferably from about 0.1 to about 3.4% by weight and particularly preferably from about 0.3 to about 2% by weight, in each case based on the weight of the colorant.

Preferred direct dyes (a) include: Basic Red 76, HC Blue 16, Basic Yellow 57, 4-hydroxypropylamino-3-nitrophenol, HC Red 13, HC Red 3, HC Blue 12, HC Orange 1, HC Blue 16 (bluequat bromide), HC Yellow 2, N,N′-bis-(2-hydroxyethyl)-2-nitro-p-phenylenediamine, 4-amino-3-nitrophenol, Ext. D&C Violet 2, HC Violet 2, Basic Brown 17, Basic Red 76 and Basic Yellow 57, and mixtures thereof. More preferred are Basic Red 76, HC Blue 16, Basic Yellow 57, 4-hydroxypropylamino-3-nitrophenol, 4-amino-3-nitrophenol, HC Red 13, HC Violet 2, and mixtures thereof. Particularly preferred are HC Blue 16, Basic Violet 2, Basic Yellow 57, 4-hydroxypropylamino-3-nitrophenol, HC Red 13 and 4-amino-3-nitrophenol and mixtures thereof.

In a preferred embodiment, the composition as contemplated herein comprises a combination of several direct dyes. By combining a variety of direct dyes, shades across the color spectrum can be created. Several purely anionic, purely cationic or purely non-ionic dyes can be combined with each other, as well as combinations of anionic, cationic and/or non-ionic dyes.

Suitable dyes for combination with each other are selected, for example, from HC Red 3, HC Blue 12, HC Orange 1, HC Blue 16 (bluequat bromide), HC Yellow 2, N,N′-bis-(2-hydroxyethyl)-2-nitro-p-phenylenediamine, Ext. D&C Violet 2, Basic Brown 17, Basic Red 76, and Basic Yellow 57.

As a further obligatory ingredient, the agents as contemplated herein contain at least one alkylamidoalkyl betaine in a total amount of about 0.05-3% by weight, preferably about 0.1-1.5% by weight, particularly preferably about 0.5-1.0% by weight, based on the weight of the agent.

Alkylamidoalkyl betaines act as amphoteric surfactants.

In a preferred embodiment, the at least one alkylamidoalkyl betaine is selected from at least one alkylamidoalkyl betaine represented by the following formula (I):

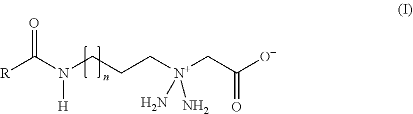

where
R represents a linear or branched, saturated or unsaturated hydrocarbon radical having about 7 to about 21, preferably about 9 to about 17 carbon atoms, further preferably about 11 to about 13, and
n is an integer from 0 to 4, in particular 1 to 2.

In a preferred embodiment, R is a linear hydrocarbon radical, in particular a linear saturated hydrocarbon radical.

In a very particularly preferred embodiment, an alkylamidoalkyl betaine of the formula (I) with R=—C9H19, —C₁₁H₂₃ or —C₁₃H₂₇ and n=0, 1 or 2 is used, in particular an alkylamidoalkyl betaine of the formula (I) with R=—C₁₁H₂₃ and n=1 is used as alkylamidoalkyl betaine (b), also known as lauramidopropyl betaine.

In an exceptionally preferred embodiment, an alkylamidoalkyl betaine of the formula (I) with n=0, 1 or 2 is used, where the radical RCO is derived from coconut fatty acids, i.e. is a mixture of n-decanoyl to n-stearoyl with a substantial proportion of n-lauroyl.

This amphoteric surfactant is also known as cocamidopropyl betaine.

However, due to the manufacturing process, common alkylamidoalkyl betaine raw materials contain a considerable amount of sodium chloride, which can be about 4.5 to about 6 wt %, based on the alkylamidoalkyl betaine solution. Surprisingly, it was found that the use of a low-sodium chloride alkylamidoalkyl betaine solution, especially a low-sodium chloride alkylamidopropyl betaine solution, can further improve the dyeing properties. Agents preferred as contemplated herein therefore contain, in each case based on their weight, sodium chloride in an amount of 0 to about 0.060% by weight, preferably about 0.005-0.040% by weight, further preferably about 0.010-0.030% by weight. Preferably, the aqueous commercial product preparation of alkylamidoalkyl betaine (b) is substantially free of sodium chloride, that is, it comprises at most about 0.6% by weight, preferably at most about 0.5% by weight, particularly preferably less than about 0.2% by weight of sodium chloride, in each case based on the total weight of the aqueous commercial product preparation of alkylamidoalkyl betaine (b). Further agents preferred as contemplated herein are exemplified in that the sodium chloride content is 0 to about 0.060% by weight, preferably about 0.005-0.040% by weight, further preferably about 0.010-0.030% by weight, in each case based on the weight of the agent as contemplated herein.

As a further obligatory ingredient, the agents as contemplated herein contain at least one nonionic ethoxylate surfactant in a total amount of about 0.2-1.5% by weight, preferably about 0.4-1.2% by weight, particularly preferably about 0.6-0.9% by weight, based on the weight of the agent.

Preferred nonionic ethoxylate surfactants are selected from ethoxylated C₈-C₂₄ alkanols with about 10-50 moles of ethylene oxide per mole, ethoxylated C₈-C₂₄ carboxylic acids with about 10-50 moles of ethylene oxide per mole and ethoxylated triglycerides with about 10-50 moles of ethylene oxide per mole, in particular ethoxylated castor oil, which may optionally be hardened or hydrogenated, and mixtures thereof.

The ethoxylated C₈-C₂₄ alkanols have the formula R¹O(CH₂CH₂O)ₙH, where R¹ is a linear or branched alkyl and/or alkenyl radical containing about 8-24 carbon atoms and n, the number of ethylene oxide units per molecule, for numbers from about 10-50, preferably about 12-30 moles ethylene oxide to 1 mole caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, Oleyl alcohol, elaidyl alcohol, petroselinyl alcohol, arachyl alcohol, gadoleyl alcohol, behenyl alcohol, erucyl alcohol and brassidyl alcohol, and technical mixtures thereof. Adducts of about 10-50 moles of ethylene oxide to industrial fatty alcohols with about 12-18 carbon atoms, such as coconut, palm, palm kernel or tallow fatty alcohols, are also suitable.

The ethoxylated C₈-C₂₄ carboxylic acids have the formula R¹(OCH₂CH₂)ₙOH, where R¹ is a linear or branched saturated or unsaturated acyl radical containing about 8-24 carbon atoms and n, the number of ethylene oxide units per molecule, for numbers from about 10-50, preferably about 12-30 moles ethylene oxide to about 1 mole caprylic acid, 2-ethylhexanoic acid, capric acid, lauric acid, isotridecanoic acid, myristic acid, cetylic acid, palmitoleic acid, stearic acid, isostearic acid, oleic acid, elaidic acid, petroselinic acid, arachidic acid, gadoleic acid, behenic acid, erucic acid and brassidic acid and their technical mixtures. Adducts of about 10-50 mol ethylene oxide to industrial fatty acids with about 12-18 carbon atoms, such as coconut, palm, palm kernel or tallow fatty acids, are also suitable. Particularly preferred are PEG-50 monostearate, PEG-50 monooleate, and PEG-50 monolaurate and mixtures thereof.

Particularly preferred are the C₁₂-C₁₈ alkanols or the C₁₂-C₁₈ carboxylic acids, each containing about 10-30 units of ethylene oxide per molecule, and mixtures of these substances, in particular ceteth-12, ceteth-20, ceteth-30, steareth-12, steareth-20, steareth-30, laureth-12 and beheneth-20, and mixtures thereof, in particular the mixture of ceteth-20 and steareth-20, also known as ceteareth-20.

Further preferred are ethoxylated triglycerides with about 10-50 moles ethylene oxide per mole, in particular ethoxylated castor oil with about 10-50 moles ethylene oxide per mole, which may optionally be hardened or hydrogenated, preferably compounds with the INCI designations PEG-40 Castor Oil and PEG-40 Hydrogenated Castor Oil.

As a further obligatory ingredient, the agents as contemplated herein contain at least one linear l-alkanol having about 8-24 carbon atoms in a total amount of about 5-15% by weight, preferably about 8-13% by weight, further preferably about 10-12% by weight, based on the weight of the agent. Preferred linear l-alkanols with about 8-24 carbon atoms are selected from lauryl alcohol, cetyl alcohol, stearyl alcohol, arachidyl alcohol, behenyl alcohol and lanolin alcohol as well as mixtures thereof, and also selected from mixtures of these alcohols as obtainable in the technical hydrogenation of vegetable and animal fatty acids, e.g. coconut alcohol. Exceptionally preferred are cetyl alcohol, stearyl alcohol and coconut alcohol, and mixtures thereof.

As a further obligatory ingredient, the agents as contemplated herein contain at least one water-soluble magnesium salt in such an amount that the content of Mg²⁺ ions is about 0.005 to about 1.000% by weight, preferably about 0.008 to about 0.500% by weight, further preferably about 0.010 to about 0.200% by weight, especially preferably about 0.013 to about 0.100% by weight, exceptionally preferably about 0.018 to about 0.050% by weight, in each case based on the weight of the agent as contemplated herein.

The content of water-soluble magnesium salt, such as magnesium chloride or trimagnesium dicitrate, surprisingly caused improved color fastness as well as at least partially improved chromaticity compared to the same formulation without water-soluble magnesium salt.

As contemplated herein, a water-soluble magnesium salt is a magnesium salt with a solubility in water of at least 5 grams per liter of solution at 20° C. Magnesium chloride, which is particularly preferred as contemplated herein, has a water solubility of 542 g/liter solution at 20° C. A compound such as magnesium carbonate with a water solubility of only 0.106 grams per liter of solution at 20° C. is therefore not a water-soluble magnesium salt within the meaning of the present disclosure.

Water-soluble magnesium salts preferred as contemplated herein are selected from magnesium chloride (95.21 g/mol, anhydrous), magnesium sulfate, magnesium bromide, magnesium benzoate, magnesium formate, magnesium acetate, magnesium propionate, magnesium nitrate, trimagnesium dicitrate (451.13 g/mol, anhydrous) and magnesium gluconate, as well as mixtures of these salts.

Other water-soluble magnesium salts preferred as contemplated herein are selected from water-soluble magnesium salts with a non-chelating anion, further preferably selected from magnesium chloride, magnesium sulfate, magnesium bromide, magnesium benzoate, magnesium formate, magnesium acetate, magnesium propionate and magnesium nitrate, and mixtures of these salts. Extremely preferred water soluble magnesium salts as contemplated herein are magnesium chloride and magnesium sulfate as well as mixtures thereof; most preferred is magnesium chloride.

The agents as contemplated herein further contain about 74-86 wt. %, preferably about 78-84 wt. %, of water, in each case based on the weight of the agent.

The total surfactant content of the agents as contemplated herein is about 0.5-6% by weight, preferably about 1-5% by weight, further preferably about 1.5-3% by weight, in each case based on the weight of the agent. This supports the hair conditioning effect of the agent as contemplated herein.

Furthermore, the agents as contemplated herein are exemplified in that no alkyl glycoside, no oxidation dye precursor and no oxidizing agent are included.

The alkyl polyglycosides excluded as contemplated herein are non-ionic surfactants exemplified by the absence of electric charges in the molecules. Alkyl polyglycosides excluded as contemplated herein are exemplified by having a hydrophobic, long-chain alkyl radical and a glycoside sugar as the hydrophilic moiety, for example laurylpolyglucoside, decylpolyglucoside, octylpolyglucoside and cocoglucoside.

The terms “surfactants” and “emulsifiers” refer to surface-active substances that can form adsorption layers on surfaces and interfaces or aggregate in bulk phases to form micell colloids or lyotropic mesophases. Basic properties of surfactants and emulsifiers are oriented absorption at interfaces as well as aggregation into micelles and the formation of lyotropic phases.

In a preferred embodiment, the agents as contemplated herein do not contain a cationic surfactant. Cationic surfactants are surfactants, i.e. surface-active compounds, each with one or more positive charges. Cationic surfactants contain only positive charges. Typically, these surfactants are composed of a hydrophobic moiety and a hydrophilic head group, with the hydrophobic moiety usually includes a hydrocarbon structure (e.g., comprising one or two linear or branched alkyl chains), and the positive charge(s) localized in the hydrophilic head group. Cationic surfactants adsorb at interfaces and aggregate in aqueous solution above the critical micelle formation concentration to form positively charged micelles.

Examples of cationic surfactants preferably excluded as contemplated herein are:
quaternary ammonium compounds which may carry one or two alkyl chains with a chain length of about 8 to about 28 carbon atoms as hydrophobic radicals,
quaternary phosphonium salts substituted by one or more alkyl chains having a chain length of about 8 to about 28 carbon atoms, or
tertiary sulfonium salts.

Furthermore, the cationic charge can also be part of a heterocyclic ring (e.g. an imidazolium ring or a pyridinium ring) in the form of an onium structure. In addition to the functional moiety carrying the cationic charge, the cationic surfactant may also contain other uncharged functional groups, as is the case, for example, with esterquats. Cationic surfactants of this type are, for example, salts of N,N,N-trimethyl-1-hexadecanaminium, especially N,N,N-trimethyl-1-hexadecanaminium chloride, salts of dimethyldistearyldimethylammonium and cationic imidazolium compounds.

In a preferred embodiment, the agents as contemplated herein do not contain an anionic surfactant. In anionic surfactants, the hydrophilic moiety includes a negatively charged hydrophilic head group. The negatively charged hydrophilic head group may be, for example, a carboxylic acid group or the salt of a carboxylic acid group, a sulfonic acid group or the salt of the sulfonic acid group, a sulfuric acid ester moiety or the salt thereof, a phosphonic acid group or the salt of the phosphonic acid group, or a phosphoric acid ester moiety or the salt thereof.

Accordingly, anionic surfactants are exemplified by the presence of a water-solubilizing anionic group such as a carboxylate, sulfate, sulfonate or phosphate group and a lipophilic alkyl group containing about 8 to about 30 carbon atoms. In addition, glycol or polyglycol ether groups, ester, ether and amide groups, and hydroxyl groups may be present in the molecule. Typical examples of anionic surfactants are alkyl benzene sulfonates, alkane sulfonates, olefin sulfonates, alkyl ether sulfonates, glycerol ether sulfonates, alpha-methyl ester sulfonates, sulfo fatty acids, alkyl sulfates, fatty alcohol ether sulfates, glycerol ether sulfates, hydroxy ether sulfates, monoglyceride (ether) sulfates, fatty acid amide (ether) sulfates, Mono- and dialkylsulfosuccinates, mono- and dialkylsulfosuccinamates, sulfotriglycerides, amide soaps, ether carboxylic acids and their salts, fatty acid isethionates, fatty acid sarcosinates, fatty acid taurates, acyl lactylates, acyl tartrates, acyl glutamates, acyl partates, alkyl oligoglucoside sulfates, protein fatty acid condensates and alkyl (ether) phosphates.

In a further, particularly preferred embodiment, the agent as contemplated herein comprises only small amounts of anionic surfactants. Preferably, the agent as contemplated herein comprises anionic surfactants in a total amount of from 0 to ≤about 0.2% by weight, more preferably from 0 to ≤about 0.15% by weight, and in particular from 0 to ≤about 0.1% by weight, based on the total weight of the agent. Very particularly, the agent is free of anionic surfactants.

In a further, particularly preferred embodiment, the agent as contemplated herein comprises as surfactants exclusively the at least one alkylamidoalkyl betaine (b) in combination with the at least one nonionic ethoxylate surfactant (c) in the amounts according to claim.

The colorants as contemplated herein may further contain additional active ingredients, auxiliaries and additives to improve the dyeing performance and to adjust further desired properties of the agents.

Dyeing processes on keratin fibers usually take place in the medium acidic to alkaline range, preferably in a weakly acidic to weakly alkaline environment. In principle, the pH of the agent can be in a range of about pH 2 to about pH 10, preferably in a range of about pH 3 to about pH 8. In order to protect the keratin fibers as far as possible and in particular to achieve a particularly clear hair-conditioning effect, the colorants as contemplated herein preferably have a pH value in the range from about 2.0 to about 7.0, preferably from about 3.5 to about pH 6.0, particularly preferably from about 4.0 to about 5.5, further preferably from about 4.1 to about 5.0 and very particularly preferably from about 4.3 to about 4.8, in each case measured at 22° C.

The pH value can be measured, for example, with a glass electrode, which is usually in the form of a combination electrode. The pH values as described herein are pH values measured at a temperature of 22° C.

The alkalizing agents which can be used to adjust the preferred pH as contemplated herein can be selected from the group formed by ammonia, alkanolamines, basic amino acids, as well as inorganic alkalizing agents such as (earth) alkali metal hydroxides, (earth) alkali metal metasilicates, (earth) alkali metal phosphates and (earth) alkali metal hydrogen phosphates. Preferred inorganic alkalizing agents are sodium hydroxide and potassium hydroxide and mixtures thereof. Organic alkalizing agents that can be used as contemplated herein are preferably selected from monoethanolamine, 2-amino-2-methylpropanol and triethanolamine. The basic amino acids which can be used as alkalizing agents as contemplated herein are preferably selected from the group formed by arginine, lysine, ornithine and histidine, particularly preferably arginine. However, it has been found in the course of the investigations relating to the present disclosure that agents which are further preferred as contemplated herein are exemplified by the fact that they additionally contain an organic alkalizing agent. One embodiment of the first subject matter of the present disclosure is exemplified in that the composition additionally contains at least one alkalizing agent selected from the group formed by ammonia, alkanolamines and basic amino acids, in particular ammonia, monoethanolamine and arginine or its compatible salts.

Acidifiers that can be used to adjust the pH are organic acids, such as citric acid, acetic acid, ascorbic acid, benzoic acid, lactic acid, malic acid and maleic acid, and mineral acids, such as hydrochloric acid, sulfuric acid or phosphoric acid.

Furthermore, it has proved advantageous if the colorants contain at least one stabilizer or complexing agent. Particularly preferred stabilizers are phenacetin, alkali benzoates (sodium benzoate) and salicylic acid. Furthermore, all complexing agents of the state of the art can be used. Complexing agents preferred as contemplated herein are nitrogen-containing polycarboxylic acids, in particular EDTA and EDDS, and phosphonates, in particular 1-hydroxyethane-1,1-diphosphonate (HEDP) and/or ethylenediaminetetramethylenephosphonate (EDTMP) and/or diethylenetriaminepentamethylenephosphonate (DTPMP) or their sodium salts.

Furthermore, the agents as contemplated herein may contain other active ingredients, auxiliaries and additives, such as non-ionic polymers, for example vinylpyrrolidinone/vinyl acrylate copolymers, polyvinylpyrrolidinone, vinylpyrrolidinone/vinyl acetate copolymers, polyethylene glycols and polysiloxanes; Silicones such as volatile or non-volatile, straight-chain, branched or cyclic, crosslinked or non-crosslinked polyalkylsiloxanes (such as dimethicone or cyclomethicone), polyarylsiloxanes and/or polyalkylarylsiloxanes, in particular polysiloxanes with organofunctional groups, such as substituted or unsubstituted amines (amodimethicones), carboxyl, alkoxy and/or hydroxyl groups (dimethicone copolyols), linear polysiloxaneA) polyoxyalkyleneB) block copolymers, grafted silicone polymers cationic polymers such as quaternized cellulose ethers, polysiloxanes with quaternary groups, dimethyldiallylammonium chloride polymers, acrylamide-dimethyldiallylammonium chloride copolymers, diethyl sulfate quaternized dimethylaminoethyl methacrylate-vinylpyrrolidinone copolymers, vinylpyrrolidinone-imidazolinium methochloride copolymers, and quaternized polyvinyl alcohol; zwitterionic and amphoteric polymers; anionic polymers such as polyacrylic acids or crosslinked polyacrylic acids; hair-conditioning compounds such as phospholipids, for example lecithin and cephalins; perfume oils, amino acids and oligopeptides; protein hydrolysates on an animal and/or vegetable basis, as well as in the form of their fatty acid condensation products or optionally anionically or cationically modified derivatives; Light stabilizers and UV blockers; active ingredients such as panthenol, pantothenic acid, pantolactone, allantoin, pyrrolidonecarboxylic acids and their salts, and bisabolol; ceramides or pseudoceramides; vitamins, provitamins and vitamin precursors; and plant extracts.

The selection of these other substances will be made by the skilled person according to the desired properties of the agents. With regard to further optional components as well as the quantities of these components used, reference is expressly made to the relevant manuals known to the skilled person. The additional active ingredients and auxiliaries are preferably present in the agents as contemplated herein in amounts of from about 0.0001 to about 2% by weight in each case, in particular from about 0.01 to about 1.5% by weight, based on the total weight of the respective agents.

The agents as contemplated herein may additionally contain further organic solvents, such as a C₁-C₄ alcohol, in particular ethanol or isopropanol, furthermore 4-methoxybutanol, ethyldiglycol, 1,2-propylene glycol n-propanol, n-butanol, 1,3-butylene glycol, glycerol, diethylene glycol monoethyl ether, and diethylene glycol mono-n-butyl ether. Preferred solvents are all water-soluble organic solvents. Preferred agents as contemplated herein are exemplified in that they additionally contain a non-aqueous solvent, preferred agents as contemplated herein containing the solvent in a concentration of about 0.1 to about 8% by weight, preferably in a concentration of about 0.5 to about 5% by weight, very preferably in a concentration of about 1 to about 3% by weight, in each case based on the total weight of the agent.

In addition, at least one penetration enhancer may also be included. Penetration enhancers can usually also act as solvents.

Examples of suitable penetration enhancers include propylene carbonate, benzyl alcohol, 2-phenoxyethan-1-ol and/or benzyl alcohol.

In another very particularly preferred embodiment, an agent as contemplated herein is therefore exemplified in that it additionally contains at least one penetration enhancer selected from the group of propylene carbonate, benzyl alcohol, 2-phenoxyethan-1-ol and/or benzyl alcohol.

Further, the agent as contemplated herein may comprise one or more cationic polymers selected from polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-14, polyquaternium-16, polyquaternium-17, polyquaternium-18, polyquaternium-22, polyquaternium-24, Polyquaternium-27, polyquaternium-28, polyquaternium-32, polyquaternium-33, polyquaternium-37, polyquaternium-39, polyquaternium-44, polyquaternium-46, polyquaternium-53, polyquaternium-55, polyquaternium-64, polyquaternium-67, polyquaternium-68, polyquaternium-69 and polyquaternium-86, and mixtures thereof, for improving the conditioning effect of the agent as well as for improving the dyeing properties.

Polyquaternium-6 is a particularly preferred cationic polymer. This improves the properties of the agent and the quality of the coloration obtained.

Preferably, at least one cationic polyquaternium compound is present in a total amount of about 0.005 to about 1% by weight, more preferably about 0.01 to about 0.8% by weight, more preferably about 0.05 to about 0.5% by weight, and in particular in an amount of about 0.1 to about 0.2% by weight, in each case based on the weight of the agent.

Particularly preferably, polyquaternium-6 is present in a total amount of 0.005 to about 1% by weight, more preferably about 0.01 to about 0.8% by weight, more preferably about 0.05 to about 0.5% by weight, and especially in an amount of about 0.1 to about 0.2% by weight, in each case based on the weight of the agent.

Furthermore, at least one silicone may preferably be included in the agents as contemplated herein to further improve the hair conditioning effect. A particularly preferred silicone is polydimethylsiloxane with the INCI dimethicone. Polydimethylsiloxanes and dimethicones suitable as contemplated herein are those with a low degree of polymerization of about 2 to about 20 as well as those with a medium degree of polymerization of about 21 to about 1000 as well as those with a degree of polymerization of more than about 1000, in particular those with a high degree of polymerization of more than about 1000 to about 25,000. The highly polymerized polydimethylsiloxanes and dimethicones also exhibit high viscosities and are present as silicone gums rather than oils. Such highly polymerized polydimethylsiloxanes and dimethicones with a degree of polymerization of more than about 1000 to about 25,000 are preferably used in pre-emulsified form as contemplated herein, particularly preferably with a number-average droplet size or particle size of about 0.05 μm to about 20 μm, preferably about 0.5 μm to about 10 μm.

Furthermore, at least one vegetable oil may preferably be included in the agents as contemplated herein to further improve the hair conditioning effect. Particularly preferred vegetable oils are selected from amaranth seed oil, apricot kernel oil, argan oil, avocado oil, babassu oil, cottonseed oil, borage seed oil, camelina oil, safflower oil, peanut oil, pomegranate seed oil, grapefruit seed oil, hemp oil, hazelnut oil, elderberry seed oil, currant seed oil, jojoba oil, linseed oil, macadamia nut oil, corn oil, Almond oil, marula oil, evening primrose oil, olive oil, palm oil, palm kernel oil, Brazil nut oil, pecan nut oil, peach kernel oil, rapeseed oil, castor oil, sea buckthorn fruit pulp oil, sea buckthorn kernel oil, sesame oil, soybean oil, sunflower oil, grape seed oil, walnut oil, wild rose oil, wheat germ oil and the liquid portions of coconut oil as well as mixtures of the aforementioned oils.

Preferably, at least one vegetable oil is present in a total amount of about 0.005 to about 1% by weight, further preferably about 0.01 to about 0.8% by weight, more preferably about 0.05 to about 0.5% by weight, and especially in an amount of about 0.1 to about 0.2% by weight, in each case based on the weight of the agent

Further preferably, marula oil is present in a total amount of about 0.005 to about 1% by weight, more preferably about 0.01 to about 0.8% by weight, more preferably about 0.05 to about 0.5% by weight, and especially in an amount of about 0.1 to about 0.2% by weight, in each case based on the weight of the agent.

The conditioner as contemplated herein can be applied directly to the keratinous fibers to be dyed, and the application method can be easily integrated into the user's everyday hygiene.

Another object of the present disclosure is therefore a method for coloring human hair, in which a cosmetic composition for the simultaneous semi-permanent coloring and care of hair, comprising

A first object of the present disclosure is a cosmetic composition for the simultaneous semi-permanent coloring and conditioning of hair according to claim 1, comprising
(a) at least one direct dye,
(b) at least one alkylamidoalkyl betaine in a total amount of about 0.05-3% by weight,
(c) at least one nonionic ethoxylate surfactant, in a total amount of about 0.2-1.5% by weight, preferably about 0.4-1.2% by weight, particularly preferably about 0.6-0.9% by weight,
(d) at least one linear l-alkanol having about 8-24 carbon atoms in a total amount of about 5-15% by weight, preferably about 8-13% by weight more preferably about 10-12% by weight,
(e) at least one water-soluble magnesium salt in such an amount that the content of Mg²⁺ ions is about 0.005 to about 1.000 wt %, preferably about 0.008 to about 0.500 wt %, more preferably about 0.010 to about 0.200 wt %, especially preferably about 0.013 to about 0.100 wt %, exceptionally preferably about 0.018 to about 0.050 wt %,
(f) about 74-86 wt % water,
   wherein no alkyl glycoside, no oxidation dye precursor and no oxidizing agent are present,
   wherein the total surfactant content is about 0.5-6 wt. %, preferably about 1-5 wt. %, further preferably about 1.5-3 wt. %, where all quantities in % by weight refer to the weight of the cosmetic product,
      is applied to the hair and rinsed out after an exposure time of about 1 minute to about 30 minutes, preferably about 5 to about 20 minutes at about 20-50° C., preferably about 20-32° C., optionally followed by drying of the hair.

With regard to further preferred embodiments of the method as contemplated herein, the same applies mutatis mutandis as to the techniques as contemplated herein.

### Examples

The following examples are intended to explain the subject matter of the present disclosure in more detail without limiting it herein.

Test Series with Nuance 6-88

The following tinting conditioners were produced. E1 and E2 are cosmetic agents as described herein, V1 and V2 are for comparison. All quantities refer to the active substance content and are in % by weight.

| | E1 | E2 | V1 | V2 |
|---|---|---|---|---|
| Coconut Alcohol | 4.9 | 4.9 | 4.9 | 4.9 |
| Cetearyl alcohol | 9.7 | 9.7 | 9.7 | 9.7 |
| Propylparaben + Methylparaben | 0.6 | 0.6 | 0.6 | 0.6 |
| Cocamidopropyl betaine | 0.7 | 0.7 | 0.7 | 0.7 |
| Ceteareth-20 | 0.9 | 0.9 | 0.9 | 0.9 |
| Monoethanolamine | 0.07 | 0.07 | 0.07 | 0.07 |
| Polyquaternium-6 | 0.2 | 0.2 | 0.2 | 0.2 |
| Etidronic acid | 0.05 | 0.05 | 0.05 | 0.05 |
| Citric acid | 0.08 | 0.08 | 0.08 | 0.08 |
| Magnesium chloride (anhydrous) | 0.1* | — | — | — |
| Trimagnesium dicitrate | — | 0.2** | — | — |
| (anhydrous) | | | | |
| Sodium chloride | 0.01 | 0.01 | 0.01 | 0.12 |
| Manila oil | 0.2 | 0.2 | 0.2 | 0.2 |
| Keratin hydrolysate with Mw | 0.02 | 0.02 | 0.02 | 0.02 |
| 400-800 Da | | | | |
| D-Panthenol | 0.2 | 0.2 | 0.2 | 0.2 |
| Cocodimonium Hydroxypropyl | 0.015 | 0.015 | 0.015 | 0.015 |
| Hydrolyzed Keratin | | | | |
| Dimethicone*** | 0.5 | 0.5 | 0.5 | 0.5 |
| Laureth-4 | 0.03 | 0.03 | 0.03 | 0.03 |
| Laureth-23 | 0.03 | 0.03 | 0.03 | 0.03 |
| Perfume | 0.7 | 0.7 | 0.7 | 0.7 |
| Butylene glycol | 3.5 | 3.5 | 3.5 | 3.5 |
| HC Red 3 | 0.15 | 0.15 | 0.15 | 0.15 |
| 4-hydroxypropylamino-3- | 0.95 | 0.95 | 0.95 | 0.95 |
| nitrophenol | | | | |
| Water @ 100.00 | 76.395 | 76.295 | 76.495 | 76.385 |

*corresponds to 0.026 wt. % Mg²⁺ ions in the tinting conditioner
**corresponds to 0.032 wt. % Mg²⁺ ions in the tinting conditioner
***ex Xiameter MEM-2664 Emulsion

All colorimetric measurements were performed using Datacolor's Spectralflash SF 450 colorimeter.

Kerling 11-0 hair strands were used for the color fastness tests.

These hair strands were each dyed with the dyeing formulations E1, E2, V1 or V2 shown above at 20° C. with 10 minutes exposure time. Then the hair strands were thoroughly rinsed with water and dried in the air stream. After dyeing and drying, the dyed hair strands were colorimetrically measured. The CIELAB values for the dyed, unwashed strands and the values C (chroma) and h (hue) calculated from them are listed in the lines “E1”, “E2”, “V1” and “V2”.

Subsequently, the dyed hair strands were washed twelve times with a commercial shampoo (Schauma 7-herb shampoo). After 6, 10 and 12 shampoo washes respectively, the hair strands were dried in an air stream and colorimetrically measured.

The color difference (ΔE) between dyed, unshampooed strand and dyed, shampooed strand was calculated according to the following formula:

ΔE=\sqrt{(Lv−Ln)²+(av−an)²+(bv−bn)²}, with

Lv, av, bv: Colorimetric values before shampoo washing

Ln, an, bn: Colorimetric values after 6, 10 or 12 shampoo washes

Chromaticity (chroma c, color direction) is calculated from the measurands a (CIE) and b (CIE) according to the following formula:

c=\sqrt{a²+b²}.

The measurand ΔE, the so-called color difference, describes the fastness of the dyeing to washing. The greater the color distance ΔE between the dyed, unshampooed strand and the dyed, shampooed strand, the greater is the color removal or washout resulting from the shampoo wash. Consequently, the lower the value of the color difference ΔE, the better the fastness to washing.

Color distances of ΔE<1 are not perceptible to the human eye. Color distances of ΔE<2 are only visible to the trained eye. Color distances of ΔE>2 are visible even to the untrained eye.

The colorants E1 and E2 as described herein differ from the comparative colorant V1 only by the addition of magnesium salt in an adequate amount. It is clearly seen that the CIE DE (ΔE) values for E1 and E2 are significantly lower than for V1 and V2, so that the addition of an adequate amount of a water-soluble magnesium salt significantly improves the color fastness.

The colorants E3 and E4 as described herein differ from the comparative colorant V3 only by the addition of magnesium salt in an adequate amount. It is clearly seen that the CIE DE (ΔE) values for E3 and E4 are significantly lower than for V3 and V4, thus the addition of an adequate amount of a water-soluble magnesium salt significantly improves the color fastness. The color intensity is also improved in both test series; this can be seen in the measured variable L, which should assume the lowest possible values.

The colorimetric data for the tests with shade 6-88 are summarized in the following table:

| Std. | Std. | Std. | Std. | Std. | Std. | |
|---|---|---|---|---|---|---|
| name | CIE L | CIE a | CIE b | CIE C | CIE h | |
| E1 | 31.18 | 35.09 | 18.74 | 39.77 | 28.10 | |
| Batch | CIE L | CIE a | CIE b | CIE C | CIE h | CIE DE |
| Name | sample | sample | sample | sample | sample | (ΔE) |
| E1 after | 42.02 | 34.68 | 19.78 | 39.93 | 29.70 | 10.90 |
| 6 washes | | | | | | |
| E1 after | 46.19 | 33.23 | 18.89 | 38.22 | 29.61 | 15.13 |
| 10 washes | | | | | | |
| E1 after | 46.61 | 30.50 | 18.10 | 35.47 | 30.68 | 16.12 |
| 12 washes | | | | | | |
| E2 | 31.70 | 36.66 | 19.57 | 41.56 | 28.10 | |
| Batch | CIE L | CIE a | CIE b | CIE C | CIE h | CIE DE |
| Name | sample | sample | sample | sample | sample | (ΔE) |
| E2 after | 39.80 | 38.04 | 21.45 | 43.68 | 29.42 | 8.43 |
| 6 washes | | | | | | |
| E2 after | 48.58 | 29.10 | 17.49 | 33.95 | 31.00 | 18.61 |
| 10 washes | | | | | | |
| E2 after | 50.34 | 30.36 | 17.63 | 35.11 | 30.14 | 19.77 |
| 12 washes | | | | | | |
| V1 | 31.49 | 36.86 | 19.64 | 41.77 | 28.05 | |
| Batch | CIE L | CIE a | CIE b | CIE C | CIE h | CIE DE |
| Name | sample | sample | sample | sample | sample | (ΔE) |
| V1 after | 45.17 | 35.11 | 19.96 | 40.39 | 29.62 | 13.80 |
| 6 washes | | | | | | |
| V1 after | 51.62 | 28.06 | 16.88 | 32.75 | 31.03 | 22.14 |
| 10 washes | | | | | | |
| V1 after | 55.57 | 23.95 | 16.41 | 29.04 | 34.42 | 27.51 |
| 12 washes | | | | | | |
| V2 | 34.80 | 38.05 | 21.14 | 43.53 | 29.06 | |
| Batch | CIE L | CIE a | CIE b | CIE C | CIE h | CIE DE |
| Name | sample | sample | sample | sample | sample | (ΔE) |
| V2 after | 47.24 | 33.97 | 19.95 | 39.40 | 30.43 | 13.14 |
| 6 washes | | | | | | |
| V2 after | 54.08 | 26.87 | 16.59 | 31.58 | 31.70 | 22.74 |
| 10 washes | | | | | | |
| V2 after | 54.62 | 24.81 | 16.01 | 29.53 | 32.83 | 24.38 |
| 12 washes | | | | | | |

Test Series with Nuance 7-65

The following tinting conditioners were produced. E3 and E4 are cosmetic agents as described herein, V3 and V4 are for comparison. All quantities refer to the active substance content and are in % by weight.

| | E3 | E4 | V3 | V4 |
|---|---|---|---|---|
| Coconut Alcohol | 4.9 | 4.9 | 4.9 | 4.9 |
| Cetearyl alcohol | 9.7 | 9.7 | 9.7 | 9.7 |
| Propylparaben + | 0.6 | 0.6 | 0.6 | 0.6 |
| Methylparaben | | | | |
| Cocamidopropyl | 0.7 | 0.7 | 0.7 | 0.7 |
| betaine | | | | |
| Ceteareth-20 | 0.9 | 0.9 | 0.9 | 0.9 |
| Polyethylene | 2.5 | 2.5 | 2.5 | 2.5 |
| glycol MG 400 | | | | |
| (PEG-8) | | | | |
| Monoethanolamine | 0.07 | 0.07 | 0.07 | 0.07 |
| Polyquaternium-6 | 0.2 | 0.2 | 0.2 | 0.2 |
| Etidronic acid | 0.08 | 0.08 | 0.08 | 0.08 |
| Citric acid | 0.08 | 0.08 | 0.08 | 0.08 |
| Magnesium | 0.10* | — | — | — |
| chloride | | | | |
| (anhydrous) | | | | |
| Trimagnesium | — | 0.2** | — | — |
| dicitrate | | | | |
| (anhydrous) | | | | |
| Sodium chloride | 0.01 | 0.01 | 0.01 | 0.12 |
| Marui a oil | 0.2 | 0.2 | 0.2 | 0.2 |
| Keratin | 0.02 | 0.02 | 0.02 | 0.02 |
| hydrolysate with | | | | |
| Mw 400-800 Da | | | | |
| D-Panthenol | 0.2 | 0.2 | 0.2 | 0.2 |
| Cocodimonium | 0.015 | 0.015 | 0.015 | 0.015 |
| Hydroxypropyl | | | | |
| Hydrolyzed keratin | | | | |
| Dimethicone*** | 0.5 | 0.5 | 0.5 | 0.5 |
| Laureth-4 | 0.03 | 0.03 | 0.03 | 0.03 |
| Laureth-23 | 0.03 | 0.03 | 0.03 | 0.03 |
| Perfume | 0.7 | 0.7 | 0.7 | 0.7 |
| HC Yellow No. 2 | 0.308 | 0.308 | 0.308 | 0.308 |
| N,N-bis(2- | 0.1995 | 0.1995 | 0.1995 | 0.1995 |
| hydroxyethyl)-2- | | | | |
| nitro-p-PD | | | | |
| HC Blue No. 12 | 0.399 | 0.399 | 0.399 | 0.399 |
| HC Red 3 | 0.0602 | 0.0602 | 0.0602 | 0.0602 |
| Hydroxyethyl-2- | 0.063 | 0.063 | 0.063 | 0.063 |
| nitro-p-toluidine | | | | |
| Water (demineral) | 77.4353 | 77.3353 | 77.5353 | 77.4253 |
| @ 100.00 wt. % | | | | |

*corresponds to 0.026 wt. % Mg²⁺ ions in the tinting conditioner
**corresponds to 0.032 wt. % Mg²⁺ ions in the tinting conditioner
***ex Xiameter MEM-2664 Emulsion

The colorimetric data for the tests with shade 7-65 are summarized in the following table:

| Std. | Std. CIE | Std. | Std. CIE | Std. CIE | Std. CIE | |
|---|---|---|---|---|---|---|
| name | L | CIE a | b | C | h | |
| Batch | CIE L | CIE a | CIE b | CIE C | CIE h | CIE DE |
| Name | sample | sample | sample | sample | sample | (ΔE) |
| E3 | 22.62 | 8.45 | 6.25 | 10.51 | 36.52 | |
| E3 after 6 | 34.32 | 11.62 | 12.21 | 16.85 | 46.42 | 13.50 |
| washes | | | | | | |
| E3 after 10 | 42.76 | 11.53 | 11.87 | 16.55 | 45.81 | 21.13 |
| washes | | | | | | |
| E3 after 12 | 44.23 | 11.80 | 11.38 | 16.39 | 43.97 | 22.45 |
| washes | | | | | | |
| E4 | 23.90 | 9.79 | 9.02 | 13.31 | 42.63 | |
| E4 after 6 | 38.26 | 11.34 | 13.32 | 17.49 | 49.59 | 15.07 |
| washes | | | | | | |
| E4 after 10 | 41.29 | 10.68 | 13.58 | 17.27 | 51.83 | 18.00 |
| washes | | | | | | |
| E4 after 12 | 47.58 | 11.38 | 10.05 | 15.19 | 41.45 | 23.76 |
| washes | | | | | | |
| V3 | 22.50 | 9.12 | 7.41 | 11.75 | 39.09 | |
| V3 after 6 | 44.35 | 11.37 | 13.21 | 17.43 | 49.28 | 22.72 |
| washes | | | | | | |
| V3 after 10 | 55.20 | 11.77 | 11.39 | 16.38 | 44.08 | 33.04 |
| washes | | | | | | |
| V3 after 12 | 56.38 | 11.97 | 11.97 | 16.93 | 45.02 | 34.30 |
| washes | | | | | | |
| V4 | 23.70 | 9.38 | 7.83 | 12.22 | 39.84 | |
| V4 after 6 | 46.19 | 11.37 | 12.19 | 16.67 | 46.98 | 22.99 |
| washes | | | | | | |
| V4 after 10 | 54.54 | 12.45 | 12.20 | 17.43 | 44.42 | 31.30 |
| washes | | | | | | |
| V4 after 12 | 55.60 | 11.76 | 11.85 | 16.69 | 45.20 | 32.24 |
| washes | | | | | | |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic agent for the simultaneous semi-permanent dyeing and care of hair, comprising:
(a) a direct dye,
(b) an alkylamidoalkyl betaine in a total amount of about 0.05 to about 3% by weight,
(c) a nonionic ethoxylate surfactant, in a total amount of about 0.2 to about 1.5% by weight,
(d) a linear l-alkanol having from about 8 to about 24 carbon atoms in a total amount of about 5 to about 15% by weight,
(e) a water-soluble magnesium salt in such an amount that the content of Mg2+ ions in the agent is from about 0.005 to about 1.000% by weight, and
(f) about 74 to about 86% by weight water,
wherein no alkyl glycosides, no oxidation dye precursors, and no oxidizing agents are present in the agent,
wherein a total surfactant content of the agent is about 0.5 to about 6% by weight, and
where all quantities in % by weight refer to a total weight of the agent.

2. The agent according to claim 1, wherein the alkylamidoalkyl betaine is an alkylamidoalkyl betaine of formula (I), 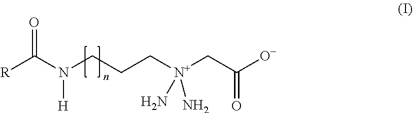
where
R represents a linear or branched, saturated or unsaturated hydrocarbon radical having 7 to about 21, carbon atoms, and
n is an integer from 0 to 4.

3. The agent according to claim 2, wherein the alkylamidoalkyl betaine of formula (I) is cocamidopropyl betaine.

4. The agent according to claim 1, wherein the alkylamidoalkyl betaine is present in a total amount of about 0.1 to about 1.5% by weight, based on the total weight of the agent.

5. The agent according to claim 1, wherein the agent is free of a cationic surfactant.

6. The agent according to claim 1, wherein the agent is free of an anionic surfactant.

7. The agent according to claim 1, wherein the agent has a pH in the range from about 2.0 to about 7.0, measured at 22° C.

8. The agent according to claim 1, wherein the water-soluble magnesium salt is selected from the group of water-soluble magnesium salts having a non-chelating anion.

9. The agent according to claim 1, wherein the direct dye is selected from the group of anionic, cationic and non-ionic direct dyes and mixtures thereof.

10. The agent according to claim 1, wherein sodium chloride is present in the agent an amount of from 0 to about 0.060% by weight, based on the total weight of the agent.

11. The agent according to claim 1, wherein the agent comprises polyquaternium 6 in an amount of about 0.005 to about 1% by weight, based on the total weight of the agent.

12. The agent according to claim 1, wherein the agent comprises a polydimethylsiloxane with the INCI dimethicone.

13. The agent of claim 1, wherein the agent comprises a vegetable oil selected from the group of amaranth seed oil, apricot kernel oil, argan oil, avocado oil, babassu oil, cottonseed oil, Borage seed oil, Camelina oil, Safflower oil, Peanut oil, Pomegranate seed oil, Grapefruit seed oil, Hemp oil, Hazelnut oil, Elderberry seed oil, Currant seed oil, Jojoba oil, Linseed oil, Macadamia nut oil, Corn oil, Almond oil, manila oil, evening primrose oil, olive oil, palm oil, palm kernel oil, Brazil nut oil, pecan nut oil, peach kernel oil, Rapeseed oil, castor oil, sea buckthorn fruit oil, sea buckthorn kernel oil, sesame oil, soybean oil, sunflower oil, Grapeseed oil, walnut oil, wild rose oil, wheat germ oil, liquid portions of coconut oil, and combinations thereof.

14. A method for coloring human hair, the method comprising the steps of:
applying a cosmetic agent to the hair, wherein the agent comprises
(a) a direct dye,
(b) an alkylamidoalkyl betaine in a total amount of from about 0.05 to about 3% by weight,
(c) a nonionic ethoxylate surfactant, in a total amount of from about 0.2 to about 1.5% by weight,
(d) a linear l-alkanol having about 8 to about 24 carbon atoms in a total amount of from about 5 to about 15% by weight,
(e) a water-soluble magnesium salt in such an amount that the content of Mg2+ ions in the agent is from about 0.005 to about 1.000% by weight, and
(f) from about 74 to about 86% by weight water,
wherein no alkyl glycosides, no oxidation dye precursors, and no oxidizing agents are present in the agent,
wherein a total surfactant content of the agent is from about 0.5 to about 6% by weight, and
where all quantities in % by weight refer to a total weight of the agent;
exposing the agent to the hair for an exposure time of from about 1 to about 30 minutes; and
rinsing the agent out of the hair.

15. The agent of claim 9, wherein the direct dye is present in the agent in an amount of from about 0.001 to about 7% by weight, based on the total weight of the agent.

16. The agent of claim 1, wherein the alkylamidoalkyl betaine is present in the agent in an amount of about 0.5 to about 1.0% by weight, based on the total weight of the cosmetic agent.

17. The agent of claim 1, wherein the agent has a pH in the range from about 4.3 to about 4.8, measured at 22° C.

18. The agent of claim 1, wherein the water-soluble magnesium salt is selected from the group of magnesium chloride, magnesium sulfate, magnesium bromide, magnesium benzoate, magnesium formate, magnesium acetate, magnesium propionate, magnesium nitrate, and mixtures thereof.

19. The agent of claim 1, wherein the water-soluble magnesium salt is magnesium chloride.

20. The agent of claim 13, wherein the vegetable oil is present in the agent in an amount of from about 0.05 to about 0.5% by weight, based on the total weight of the agent.
